(19) United States
(12) Patent Application Publication
(10) Pub. No.: US 2011/0125153 A1
(43) Pub. Date: May 26, 2011

(54) INTRAMEDULLARY FIXATION ASSEMBLY AND METHOD OF USE

(76) Inventors: Jeff Tyber, Bethlehem, PA (US); Jamy Gannoe, West Milford, NJ (US)

(21) Appl. No.: 12/818,483

(22) Filed: Jun. 18, 2010

Related U.S. Application Data
(63) Continuation-in-part of application No. 12/460,069, filed on Jul. 13, 2009.
Application No. 12/460,069 is a continuation-in-part of application No. 12/456,808, filed on Jun. 23, 2009.
(60) Provisional application No. 61/132,932, filed on Jun. 24, 2008.

(51) Int. Cl. A61B 17/58

(52) U.S. Cl. 606/62

(57) ABSTRACT

An intramedullary fixation assembly for bone fusion includes an implant member positioned at a proximal end of the intramedullary fixation assembly, a lag screw member positioned at a distal end of the intramedullary fixation assembly and a circular member frictionally coupled to the lag screw member. The lag screw member is slideably coupled to the implant member and provides for an interference fit with the implant member.

### CROSS-REFERENCE TO RELATED APPLICATIONS

This application is a continuation-in-part application of Non-Provisional application Ser. No. 12/460,069, filed Jul. 13, 2009, which claims the benefit of Non-Provisional Application No. 12/456,808, filed Jun. 23, 2009, which claims the benefit of Provisional Application No. 61/132,932, filed Jun. 24, 2008, the entire contents of the chain of application is herein incorporated by reference.

### FIELD OF THE INVENTION

This invention relates to the field of orthopedic implant devices, and more particularly, to an intramedullary fixation assembly used for fusion of the angled joints, bones and deformity correction, such as the metatarsal and phalangeal bones in the foot.

### BACKGROUND OF THE INVENTION

Orthopedic implant devices, such as intramedullary nails, plates, rods and screws are often used to repair or reconstruct bones and joints affected by trauma, degeneration, deformity and disease, such as Charcot arthropathy caused by diabetes in some patients, Hallux Valgus deformities, failed Keller Bunionectomies, Rheumatoid Arthritis, and severe deformities. Charcot arthropathy (or Charcot foot) is a destructive process affecting many regions including joints of the foot and ankle in diabetics. This condition causes bony fragmentation, dislocation, and fractures that eventually progresses to foot deformity, bony prominences, ulceration and instability of the foot. Charcot arthropathy can affect any joint in the body but is often seen in the feet affecting the metatarsal, tarsometatarsal and tarsal joints and frequently causes the foot to lose its arch or curvature, thus resulting in “flat footedness” in the mid-foot region.

Early treatment for Charcot foot includes the use of therapeutic footwear, immobilization of the foot and/or non-weight bearing treatment. Surgical treatments include orthopedic fixation devices that fixate the bones in order to fuse them into a stable mass. These orthopedic implant devices realign bone segments and hold them together in compression until healing occurs, resulting in a stable mass.

In order to restore an arch in a Charcot foot, the physician must estimate the arch and manually align the bones and deliver the screws to hold the bones in place, while reducing bone purchase. Intramedullary nails and/or a plate with a lag screw too have deficiencies. These intramedullary nails also do not reconstruct an arch that is lost due to Charcot foot disease.

Moreover, infections and wound complications are a major concern in the aforementioned procedures. Wound closure is technically demanding for the surgeon, and devices that add surface prominence, such as plates or exposed screws, add to the difficulty by requiring greater tissue tension during incision reapproximation. This increases the risk of postoperative wound infections and dehiscence that may ultimately result in limb amputation.

Various implants have been utilized for surgical treatment of these bones and joints, including bone screws. Implants have also been utilized to treat severe deformities in the metatarsal and phalangeal bones, including multiple screws and plates. These multiple screws and plate implants have been commonly used in a first metatarsal-phalangeal fusion procedure to fuse the first metatarsal to the first phalangeal bone in hallux valgus deformities, failed keller bunionectomies, rheumatoid arthritis, and other types of severe deformities in the metatarsal and phalange bones. While these devices allow fixation and promote fusion, they do not deliver restoration of the arch in a Charcot foot nor are they effective in metatarsal-phalangeal (MTP) fusion procedures.

Particularly, screw implants in MTP procedures are ineffective in delivering sufficient compression to the bones in the foot, preventing screw head break out, or delivering effective bending resistance. Moreover, hard to control dorsiflexion and valgus angles as well skin irritation from proximity to the skin prevents these screw implants from being readily utilized for surgical treatment. Yet further, plate implants used with bone screws too have the same drawbacks as fixed varus and valgus angles, lack of direct compression across the MTP joint, and skin irritations from proximity to the skin reduce the effectiveness of these implants.

There is therefore a need for an intramedullary fixation assembly and method of use that overcomes some or all of the previously delineated drawbacks of prior fixation assemblies.

### SUMMARY OF THE INVENTION

An object of the invention is to overcome the drawbacks of previous inventions.

Another object of the invention is to provide a novel and useful intramedullary fixation assembly that may be utilized to treat bones in a mid-foot and forefoot regions.

Another object of the invention is to restore the arch by utilizing an intramedullary assembly.

Another object of the invention is to provide a system for treating deteriorating bones in a mid-foot region.

Another object of the invention is to provide a method for restoring the arch of the foot by delivering a fixator that can be coupled in a patient's foot.

Another object of the invention is to fuse the metatarsal phalangeal joint by utilizing an intramedullary assembly.

In a first non-limiting aspect of the invention, a fixation assembly comprising two members is provided. A first member, positioned at a proximal end of the fixation assembly, has an elongated portion and a tapered bulbous end. A second member, positioned at a distal end of the fixation assembly, has an internal tapered aperture, wherein the elongated portion resides within the internal tapered aperture. The first member forms a fixed angle with the second member, thereby selectively coupling the first member to the second member.

In a second non-limiting aspect of the invention, a method for reconstructing an arch in a mid-foot region comprises eight steps. Step one includes making an incision in the mid-foot region of a patient's foot. Step two includes gunstocking the foot to expose the articular surface. Step three includes reaming the intramedullary canal and inserting a distal member. Step four includes coupling the instrument to the distal member. Step five includes assessing the position of the proximal member with a guide wire. Step six includes pre-drilling a hole through the joints selected for fusion. The seventh step includes inserting the proximal member over the guide wire until rigid connection with the tapered aperture is made that compresses the joint and wherein the proximal member is at an angle to the distal member. The eighth step includes removing the instrument and closing the incision, thereby causing the arch to be formed in the mid-foot region.

In a third non-limiting aspect of the invention, an instrument is combined with a fixation assembly for reconstructing an arch in a mid-foot region. The instrument has a handle, a “U-shaped” recess having two sides and a tapered bore. The intramedullary fixation assembly has a first member and a second member. The first member is positioned at a proximal end of the intramedullary fixation assembly. The first member has an elongated portion and a bulbous portion. The second member is positioned at a distal end of the intramedullary fixation assembly. The second member has an internal tapered aperture, a plurality of grooves and a threaded portion. The elongated portion resides within the internal tapered aperture, and a “U-shaped” recess having two sides that couple the first member to the second member, and further coupling the instrument to the intramedullary fixation assembly for reconstructing the arch in the mid-foot region.

In a fourth non-limiting aspect of the invention, an intramedullary fixation assembly for bone fusion includes a first member positioned at a proximal end of the intramedullary fixation assembly, and a second member positioned at a distal end of the intramedullary fixation assembly. The first member is slideably coupled to the second member and provides for an interference fit with the second member.

In a fifth non-limiting aspect of the invention, a method for fusing a metatarsal phalangeal joint comprises seven steps. Step one includes providing a fixation assembly, where the fixation assembly includes a metatarsal implant member for connecting to a metatarsal bone and a phalangeal implant member for connecting to a phalange bone. Step two includes drilling the metatarsal medullary canal and drilling the phalangeal medullary canal. Step three includes inserting the metatarsal implant member into the metatarsal medullary canal. Step four includes inserting the phalangeal implant member into the internal aperture of the metatarsal implant member. Step six includes inserting the phalangeal implant member into the phalangeal medullary canal. Step seven includes applying compression to the phalangeal implant member to lock the metatarsal implant member to the phalangeal implant member, thereby fusing the metatarsal phalangeal joint.

In a sixth non-limiting aspect of the invention, a fixation assembly for a metatarsal-phalangeal fusion in a human foot includes a metatarsal implant member for connecting to a metatarsal bone, where the metatarsal implant member is positioned at a proximal end of the fixation assembly, and a phalangeal implant member for connecting to a phalange bone, where the phalangeal implant member is positioned at a distal end of the fixation assembly. The phalangeal implant member is slideably coupled to the metatarsal implant member and provides for an interference fit with the metatarsal implant member.

In a seventh non-limiting aspect of the invention, a fixation assembly for a metatarsal-phalangeal fusion in a human foot includes an implant member positioned at a proximal end of the intramedullary fixation assembly, a lag screw member positioned at a distal end of the intramedullary fixation assembly and a circular member frictionally coupled to the lag screw member. The lag screw member is coupled to the implant member at a variable angle and locks the lag screw member to the implant member when compression is applied by the intramedullary fixation assembly on bone fragments.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of a fixation system according to a preferred embodiment of the invention.

FIG. 2 is a perspective view of a proximal screw member used in the fixation system shown in FIG. 1 according to the preferred embodiment of the invention.

FIG. 3A is a perspective view of a distal member used in the fixation system shown in FIG. 1 according to the preferred embodiment of the invention.

FIG. 3B is a perspective cross-sectional view of the distal member shown in FIG. 3A according to the preferred embodiment of the invention.

FIG. 4 is a perspective view of the instrument member used in the fixation system shown in FIG. 1 according to the preferred embodiment of the invention.

FIG. 5 is a perspective view of the assembled intramedullary fixation assembly inserted into the bones of a patient's foot according to the preferred embodiment of the invention.

FIG. 6 is a side view of the assembled intramedullary fixation assembly shown in FIG. 5 according to the preferred embodiment of the invention.

FIG. 7 is a flow chart illustrating the method of coupling the intramedullary fixation assembly shown in FIGS. 1-6 to tarsal and metatarsal bones in a patient's foot according to the preferred embodiment of the invention.

FIG. 8 is a perspective view of an assembled intramedullary fixation assembly inserted into the metatarsal and trapezial bones of a patient's foot according to an alternate embodiment of the invention.

FIG. 9 is a perspective cross-sectional view of the intramedullary fixation assembly according to the alternate embodiment of the invention.

FIG. 10A is a perspective view of a metatarsal implant member used in the intramedullary fixation assembly shown in FIG. 8 according to the alternate embodiment of the invention.

FIG. 10B is a perspective cross-sectional view of the metatarsal implant member used in the intramedullary fixation assembly shown in FIG. 10A according to the alternate embodiment of the invention.

FIG. 11 is a perspective cross-sectional view of a phalangeal implant member used in the intramedullary fixation assembly shown in FIG. 8 according to the alternate embodiment of the invention.

FIG. 12 is a perspective cross-sectional view of a locking set screw used in the intramedullary fixation assembly shown in FIG. 8 according to the alternate embodiment of the invention.

FIG. 13 is a flow chart illustrating the method of coupling the intramedullary fixation assembly shown in FIGS. 8-12 to metatarsal and phalangeal bones in a patient's foot according to the alternate embodiment of the invention.

FIG. 14 is a perspective view of the assembled intramedullary fixation assembly inserted into the bones of a patient's foot according to an alternate embodiment of the invention.

FIG. 15 is a perspective view of the intramedullary fixation assembly shown in FIG. 14 according to the alternate embodiment of the invention.

FIG. 16 is a perspective view of a metatarsal implant member used in the intramedullary fixation assembly shown in FIGS. 14-15 according to the alternate embodiment of the invention.

FIG. 17 is a perspective view of a phalangeal implant member used in the intramedullary fixation assembly shown in FIGS. 14-15 according to the alternate embodiment of the invention.

FIG. 18 is a perspective view of an assembled intramedullary fixation assembly according to an alternate embodiment of the invention.

FIG. 19A is a perspective view of a metatarsal implant member used in the intramedullary fixation assembly shown in FIG. 18 according to the alternate embodiment of the invention.

FIG. 19B is a cross-sectional view of the metatarsal implant member used in the intramedullary fixation assembly shown in FIG. 18 according to the alternate embodiment of the invention.

FIG. 20 is a perspective view of a lag screw member used in the intramedullary fixation assembly shown in FIG. 18 according to the alternate embodiment of the invention.

FIG. 21 is a perspective cross-sectional view of a spherical ring used in the intramedullary fixation assembly shown in FIG. 18 according to the alternate embodiment of the invention.

FIG. 22 is a flow chart illustrating the method of coupling the intramedullary fixation assembly shown in FIG. 18-21 to the metatarsal and phalangeal bones in a patient's foot according to the alternate embodiment of the invention.

### DETAILED DESCRIPTION OF THE INVENTION

The invention may be understood more readily by reference to the following detailed description of preferred embodiment of the invention. However, techniques, systems and operating structures in accordance with the invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

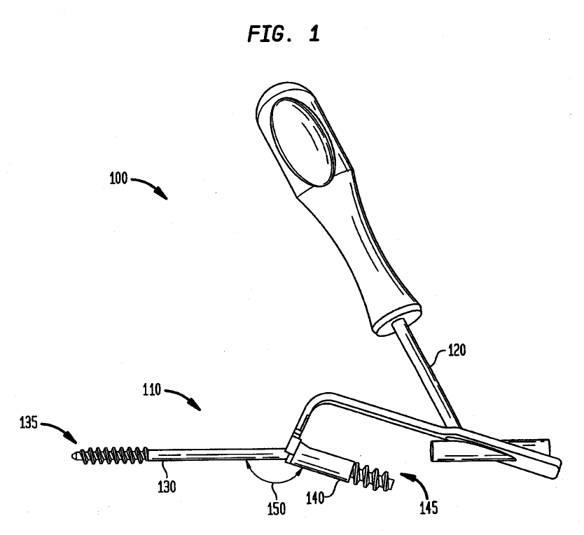

Referring now to FIG. 1, there is shown a fixation system 100 which is made in accordance with the teachings of the preferred embodiment of the invention. As shown, the fixation system 100 includes an intramedullary fixation assembly 110, comprising a proximal screw member 130 and a distal member 140. Proximal screw member 130 is provided on proximal end 135 of assembly 110 and is coupled to a distal member 140 that is provided on the distal end 145 of the fixation assembly 110. Also, proximal screw member 130 makes a fixed angle 150 with distal member 140 and this angle 150 determines the angle for arch restoration. Moreover, fixation system 100 includes instrument 120 that is utilized to couple intramedullary fixation assembly 110 to the bones in the mid-foot region (not shown). It should be appreciated that in one non-limiting embodiment, intramedullary fixation assembly 110 may be made from a Titanium material, although, in other non-limiting embodiments, intramedullary fixation assembly 110 may be made from SST, PEEK, NiTi, Cobalt chrome or other similar types of materials.

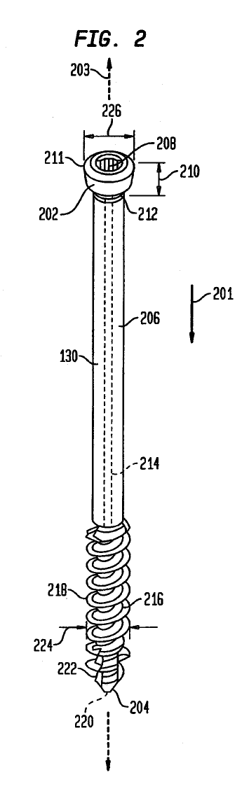

As shown in FIG. 2, proximal screw member 130 is generally cylindrical in shape and extends from first bulbous portion 202 to second tapered end 204. End 204 has a diameter that is slightly smaller than diameter 226 of bulbous portion 202. Additionally, bulbous portion 202 has a taper, such as a Morse taper, with a width that decreases from end 211 to end 212. The taper allows for a locked interference fit with tapered aperture 316 when tapered bulbous portion 202 is combined with tapered aperture 316, shown and described below. Moreover, bulbous portion 202 is generally circular and has a generally hexagonal torque transmitting aperture 208 that traverses length 210 of bulbous portion 202. However, a star-shaped aperture, a square-shaped aperture, or any other shaped aperture may be utilized without departing from the scope of the invention. Torque transmitting aperture 208 is utilized to transmit a torque from bulbous portion 202 to tapered end 204 by rotating bulbous portion 202.

Further, proximal screw member 130 has a first smooth exterior portion 206 extending from end 212 of bulbous portion 202. Portion 206 comprises an internal aperture 214 that longitudinally traverses portion 206 in direction 201. Portion 206 terminates into a second generally tubular portion 216. Portion 216 may comprise internal circular aperture 220 that longitudinally traverses inside portion 216. Internal circular aperture 220 is aligned with apertures 214 and 208 along axis 203 to form a continuous opening (i.e., a cannula) from bulbous portion 202 to end 204. The continuous opening or cannula is provided to interact with a guide wire (not shown) by receiving the guide wire within the continuous opening thereby positioning and locating the proximal member 130. In other non-limiting embodiments, the proximal member 130 may be provided without apertures 220 and 214 (i.e., the proximal member is solid).

Furthermore, tubular portion 216 has a plurality of circular threads, such as threads 218, which are circumferentially disposed on the external surface of portion 216 and, with threads 218 having an external diameter 224. Portion 216 may also be provided with a self-tapping leading edge 222 to provide portion 216 with the ability to remove bone material during insertion of proximal screw member 130 into bone. It should be appreciated that the length of the proximal member 130 may be selected of varying lengths to allow a surgeon to fuse different joints in a foot (not shown).

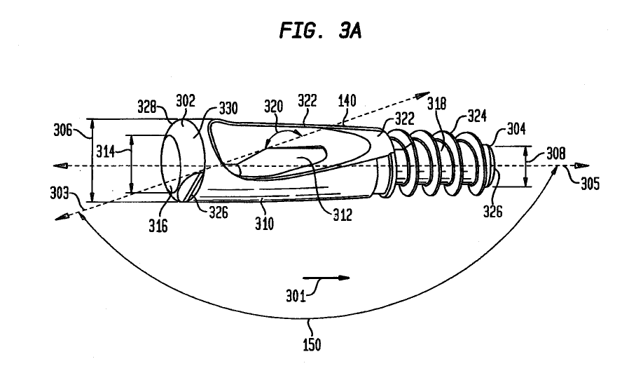

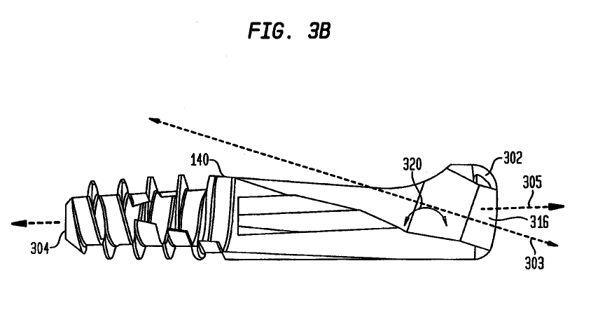

As shown in FIGS. 3A-3B, distal member 140 of the preferred embodiment is generally tubular in shape and tapers from a first end 302 to a second end 304 (i.e. end 302 has a diameter 306 that is slightly larger than diameter 308 of end 304). However, in another non-limiting embodiment, distal member 140 has a constant width from first end 302 to second end 304. Further, first end 302 is generally semi-spherical in shape and has an internal circular aperture 316, which traverses end 302 along direction 301 (i.e. end 302 is generally “donut” shaped). Additionally, circular aperture 316 emanates from surface 322, such that portion 310 has a generally tapered aperture 316 provided in portion 310. Circular aperture 316 comprises slope 320 from first end 302 to end 322 of portion 310. Further, aperture 316 is aligned along axis 303, which is offset from horizontal axis 305 of distal member 140. Axis 303 forms an angle 150 with horizontal axis 305 that determines the angle for arch restoration, as shown in FIG. 3A. Angle 150 may be any angle greater than 90 degrees and less than 180 degrees. Tapered aperture 316 when combined with tapered bulbous portion 202, shown in FIG. 2, creates a locked interference fit between proximal member 130 and distal member 140. First end 302 has a plurality of substantially similar grooves 326 and 328, which form an “L-shape” with surface 330 of end 302. Grooves 326 and 328 are provided to receive instrument 120 of fixation system 100, which is later described. In other non-limiting embodiments, other similar instruments may be provided to be received within grooves 326 and 328.

Distal member 140 further comprises a generally smooth portion 310 coupled to end 302. Portion 310 has a generally hexagonal shaped aperture 312, which opens into aperture 316 and which longitudinally traverses through portion 310 in direction 301. In other non-limiting embodiments, a star-shaped aperture, a square-shaped aperture, or any other shaped aperture may be utilized. Circular aperture 316 has a diameter 314 that is slightly larger than external diameter 224 of portion 216 and 206 of proximal screw member 130, with portions 216 and 206 being slidably received within aperture 316 of portion 310. Aperture 316 has a diameter that is smaller than diameter 226 of bulbous portion 202.

Portion 310 of distal member 140 terminates into a second generally cylindrical portion 318 which has a plurality of threads 324, which are circumferentially disposed on the external surface of portion 318. Portion 318 has an internal circular aperture 326 which is longitudinally coextensive with portion 318 in direction 301. Circular aperture 326 aligns with aperture 312 to form a continuous opening from end 302 to end 304.

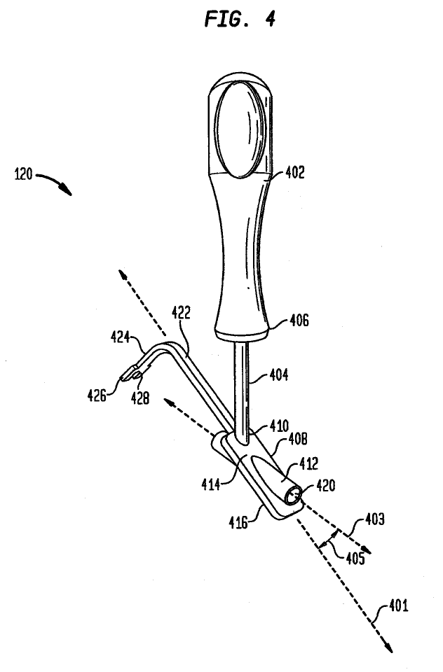

As shown in FIG. 4, instrument 120 is illustrated for coupling proximal screw member 130 to distal member 140. Particularly, instrument 120 includes a handle portion 402 coupled to a rod portion 404. Rod portion 404 emanates from handle portion 402 at end 406 and terminates into a rectangular planar portion 408 at end 410. Planar portion 408 is aligned along axis 401 and is fixably coupled to a generally cylindrical tubular portion 412 (i.e., an aiming device). Portion 412 traverses portion 408 from top surface 414 to bottom surface 416. Further, tubular portion 412 is aligned along dissimilar axis 403, forming an angle 405 with axis 401. Also, tubular portion 412 has a through aperture 420 that longitudinally traverses portion 412 along axis 403.

Planar portion 408 is coupled to planar portion 422, with portion 422 having a width slightly smaller than width of portion 408. Portion 422 terminates into a generally “U-shaped” portion 424 with portion 424 being orthogonal to portion 422. Further, portion 424 has a plurality of substantially similar sides 426 and 428 which are provided to be slidably coupled to grooves 326 and 328 of distal member 140.

In operation, sides 426 and 428 of instrument 120 are received in respective grooves 326 and 328 of distal member 140, of FIGS. 3A-3B, thereby slidably coupling distal member 140 to instrument 120. In this position, axis 303 of aperture 316 is aligned along substantially the same axis as axis 403 of instrument 120. Proximal screw member 130 is coupled to distal member 140 by slidably coupling portions 206 and 216 through aperture 420 of tubular portion 412. Tubular portion 412 guides proximal screw member 130 through internal aperture 420 and into aperture 316 on surface 322 and may also guide a Kirschner wire (K wire) or a drill. Proximal screw member 130, of FIG. 2, travels into bone as portions 216 and 206 travel further through aperture 316 at end 302 until bulbous portion 202 is restrained by surface 322 and end 302. Aperture 316, being tapered along axis 303, causes proximal screw member 130 to form an angle 150 with distal member 140, with proximal member 130 being aligned along an axis 303, which is substantially the same axis as axis 403 of tubular portion 412 of instrument 120.

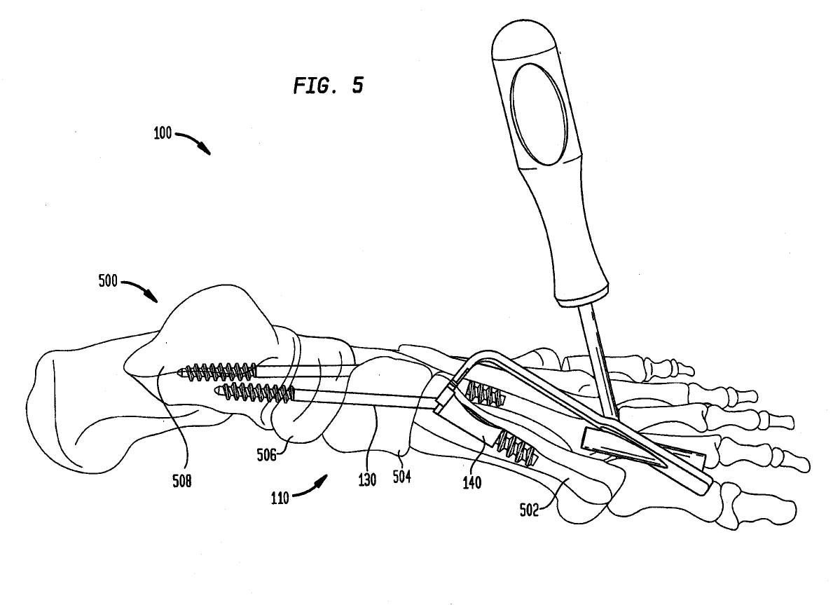

In operation, and as best shown in FIGS. 5, 6 and 7, the fixation system 100 utilizes the intramedullary fixation assembly 110 for treating and fixating the deteriorated and damaged or fractured bones in the human foot 500. This restores the arch in a human foot 500 by coupling the intramedullary fixation assembly 110 to the human foot 500 of a left leg. In one-non limiting example, and as shown in FIG. 5, the intramedullary assembly 110 is coupled to the medullary canals of the first metatarsal 502, medial cuneiform 504, navicular 506 and talus bone 508. Talus bone 508 makes up part of the ankle joint where the threaded portion 216 of the proximal screw member 130 of the intramedullary assembly 110 is threadably coupled. The medial cuneiform 504 and navicular 506 bones are most affected by Diabetic Charcot foot disorder that causes deterioration and collapse of the arch of the foot 500. It should be appreciated that the intramedullary assembly 110 may be used within each of the five rays, with a ray representing a line drawn from each metatarsal bone to the talus. The angulation in the smaller rays will be smaller than the two rays (i.e., a line from the first and second metatarsal bones to the talus bone). Also, the diameter of distal member 140 will decrease from the large ray to the small ray. In one non-limiting example, the angulation may be any angle greater than 90 degrees and less than 180 degrees. For example, the angle for the first ray may be 150-170 degrees and the angles for the other rays may be 160-175 degrees.

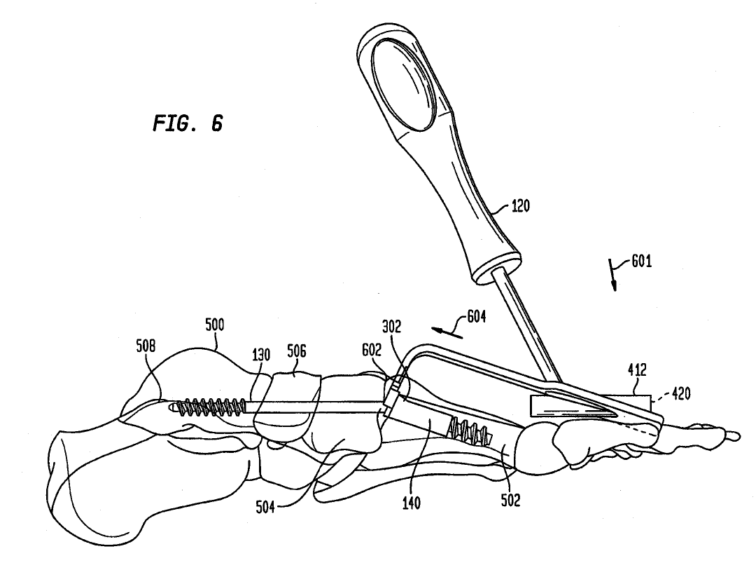

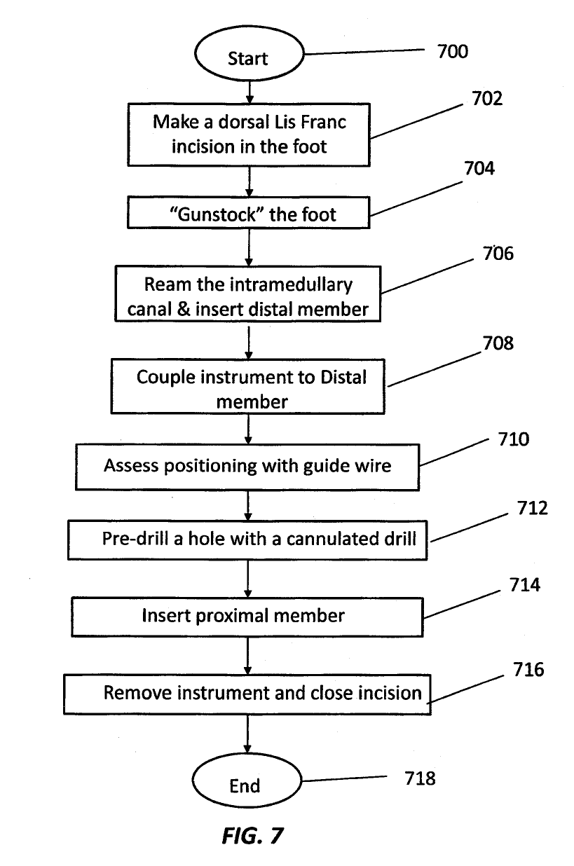

As shown in FIGS. 6 and 7, the intramedullary fixation assembly 110 may be utilized to reconstruct an arch in a mid-foot region of a human foot 500. As shown, the method starts in step 700 and proceeds to step 702, whereby a Dorsal Lis Franc incision (i.e., mid-foot incision) (not shown) is made in foot 500 in order to gain access to the joint. In step 704, the joint capsule is separated by “Gunstocking” foot 500 in direction 601 (i.e., the foot 500 is bent mid-foot) to expose the articular surface 602 and the articulating cartilage is removed. Next, in step 706, the intramedullary canal is reamed and the distal member 140 is inserted into the intramedullary canal (not shown) of the metatarsal 502. In other non-limiting embodiments, the distal member 140 may be inserted by impaction, by press fit, by reaming a hole in the intramedullary canal (not shown) or substantially any other similar strategy or technique.

Next, in step 708, the instrument 120 is coupled to the distal member 140 by coupling sides 426 and 428 of instrument 120 to respective grooves 326 and 328. In step 710, initial positioning of the proximal member 130 is assessed with the use of a guide wire through portion 412 (i.e., aiming device). Next, in step 712, a countersink drill is inserted through portion 412 and the proximal cortex is penetrated. In this step, a cannulated drill or guide wire is used to pre-drill the hole through the joints selected for fusion. In step 714, the proximal screw member 130 is inserted over the guide wire and into the distal member 140. Particularly, the proximal member 130 is inserted through tubular portion 412 (i.e., aiming device), causing proximal member 130 to travel through internal longitudinal aperture 420, into distal member 140 and further into bones 504, 506 and 508 until rigid connection with the tapered aperture 316 is made, thereby compressing the joint. In one non-limiting embodiment, a locking element (not shown) such as a plate or a washer is coupled to end 302 of the intramedullary fixation assembly 110 to further secure proximal threaded member 130 to distal member 140. Next, in step 716 the instrument 120 is removed and the dorsal Lis Franc (i.e., mid-foot) incision is closed. The method ends in step 718.

It should be appreciated that a plurality of intramedullary fixation assemblies, such as intramedullary fixation assembly 110, may be inserted into any of the bones of a foot 500 such as, but not limited to the metatarsal, cuneiform, calcaneus, cuboid, talus and navicular bones, in order to restore the natural anatomical shape of the arch of the foot 500. Thus, the fixation system 100, in one non-limiting embodiment, is utilized to couple the intramedullary fixation assembly 110 to the foot 500, which causes the metatarsal 504, medial cuneiform 504, navicular 506 and talus 508 bones to be aligned to the proper anatomical shape of an arch when assembled within foot 500. It should be appreciated that the intramedullary fixation assembly 110 is delivered through a dorsal midfoot incision, thereby reducing the disruption to the plantar tissues and/or the metatarsal heads while at the same time minimizing the tension on the skin. This allows for improved wound closure, reduced operating room time, reduction in the number of incisions required and reduction in the total length of incisions. It should also be appreciated that in other non-limiting embodiments, the intramedullary assembly 110 may be utilized with graft material (i.e., autograft, allograft or other biologic agent).

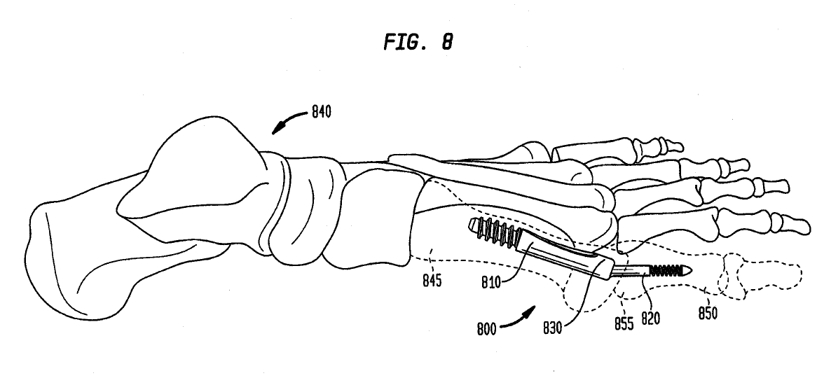

In an alternate embodiment, as shown in FIG. 8, an intramedullary fixation assembly 800 may comprise three interconnected members for the internal fixation of the first metatarsal 845 to the first proximal phalange 850 in the human foot 840 or any other appropriate use for the internal fixation of the other bones in the human foot 840. The interconnected members of the intramedullary fixation assembly 800 may be inserted into the medullary canals of the first metatarsal 845 and the first proximal phalange 850 in order to restore the angle in the toes of a human foot 840. Particularly, the intramedullary fixation assembly 800 may comprise a metatarsal implant member 810, a phalangeal implant member 820 and an optional locking set screw 830.

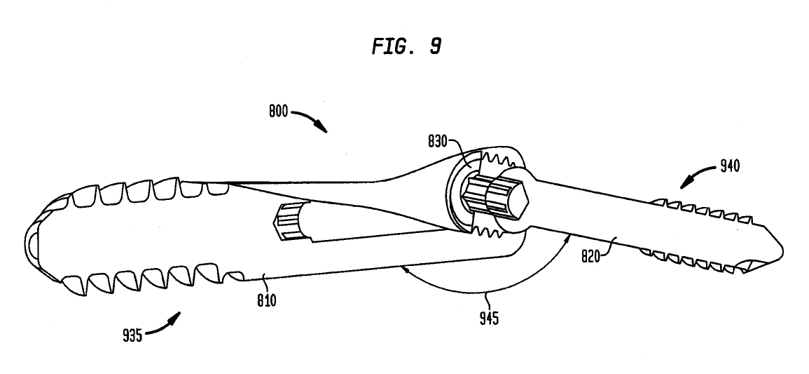

As shown in FIG. 9, the intramedullary fixation assembly 800 comprises the metatarsal implant member 810, which is provided on the distal end 935 of the intramedullary fixation assembly 800, the phalangeal implant member 820 provided on the proximal end 940 and the optional locking set screw 830 provided to threadably couple to the metatarsal implant member 810, thereby pressure coupling the metatarsal implant member 810 to the phalangeal implant member 820. Optional locking set screw 830 thereby locks the metatarsal implant member 810 to the phalangeal implant member 820 and allows for incremental adjustment of the position of phalangeal implant member 820 within the metatarsal implant member 810 as will be shown and described.

In its implanted position, metatarsal implant member 810 is at a fixed angle 945 with phalangeal implant member 820 and this angle 945 may be adjusted in order to set the angle for restoration. It should be appreciated that in one non-limiting embodiment, intramedullary fixation assembly 800 may be made from a Titanium material, although, in other non-limiting embodiments, intramedullary fixation assembly 800 may be made from SST, PEEK, NiTi, Cobalt chrome or other similar types of materials.

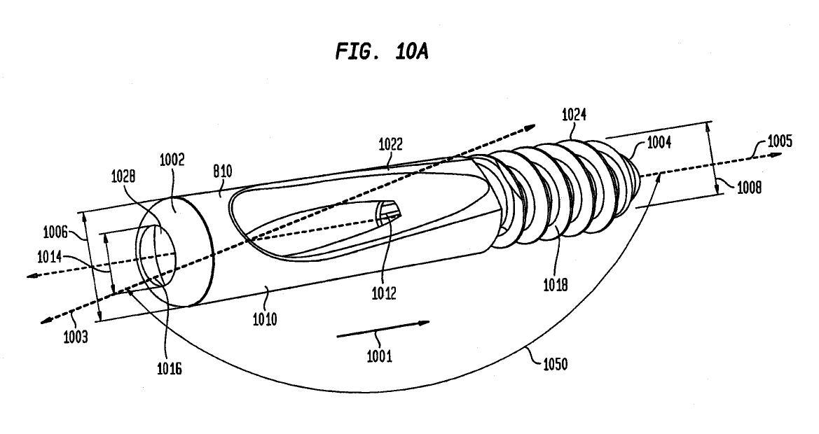

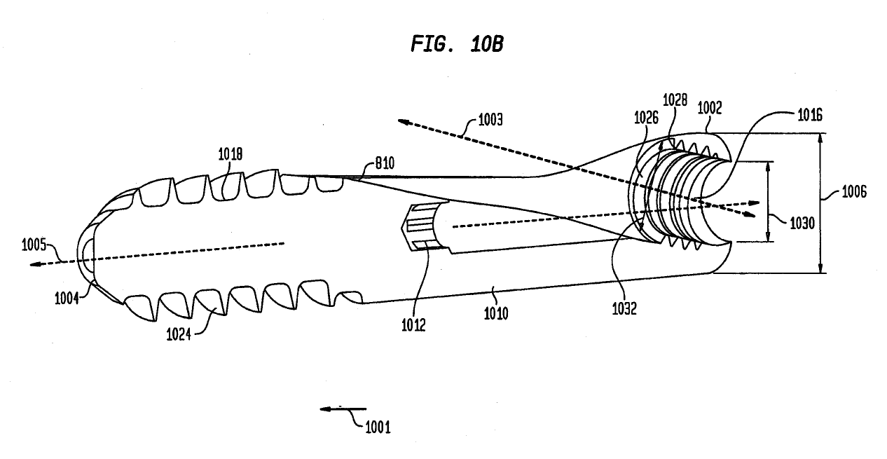

As shown in FIGS. 10A-10B, metatarsal implant member 810 of the embodiment is generally tubular in shape and tapers from a first end 1002 to a second end 1004 (i.e. end 1002 has a diameter 1006 that is slightly larger than diameter 1008 of end 1004). However, in another non-limiting embodiment, metatarsal implant member 810 has a constant width from first end 1002 to second end 1004. Further, first end 1002 is generally semi-spherical in shape and has an internal circular aperture 1016, which traverses end 1002 along direction 1001 (i.e. end 1002 is generally “donut” shaped). Additionally, circular aperture 1016 is aligned along axis 1003, which is offset from horizontal axis 1005 at an angle 1050. Angle 1050 causes circular aperture 1016 to emanate from surface 1022, such that circular aperture 1016 on cylindrical portion 1010 is generally tapered with the diameter 1030 being slightly smaller than diameter 1032.

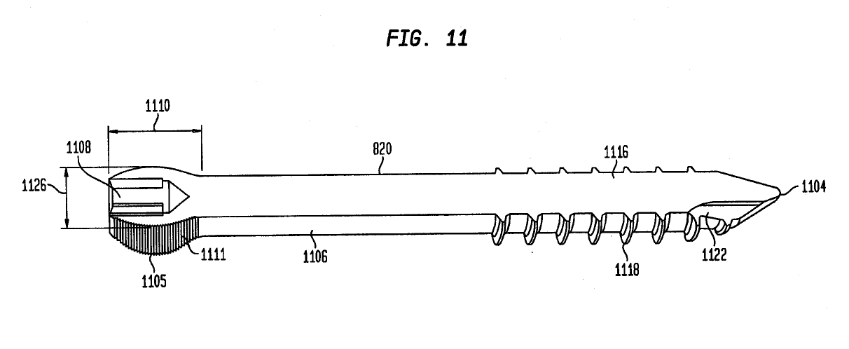

It should be appreciated that angle 1050 initially determines the angle for restoration, as shown in FIG. 10A. Angle 1050 may be any angle greater than 90 degrees and less than 180 degrees. Circular aperture 1016 also includes a plurality of substantially similar threads 1028 that are provided on interior surface 1026 of metatarsal implant member 810. The plurality of substantially similar threads 1028 when combined with head portion 1105 of phalangeal implant member 820, shown in FIG. 11, creates a locked interference fit between metatarsal implant member 810 and phalangeal implant member 820. Circular aperture 1016 has a diameter 1014 that is provided to receive head portion 1105 of phalangeal implant member 820, which will be shown in FIG. 11.

Also, metatarsal implant member 810 further comprises a generally smooth portion 1010 coupled to end 1002. Portion 1010 has a generally hexagonal shaped aperture 1012 aligned along axis 1005.

In other non-limiting embodiments, a star-shaped aperture, a square-shaped aperture, or any other shaped aperture may be utilized. Aperture 1012 emanates from circular aperture 1016, traverses through portion 1010 in direction 1001 and terminates into a generally cylindrical portion 1018. In other non-limiting embodiments, aperture 1012 is longitudinally coextensive with portion 1018 in direction 1001 and emanates from second end 1004. In this manner, a continuous opening from end 1002 to end 1004 may be formed to receive a Kirschner wire (K wire) or a drill.

Further, portion 1018 has a plurality of substantially similar circumferential threads, such as threads 1024, which are circumferentially disposed on the external surface of portion 1018. It should be appreciated that plurality of circumferential threads, such as threads 1024, are provided so that rotating metatarsal implant member 810 causes the plurality of circumferential threads 1024 to grip or catch the medullary canal of first metatarsal 845 (shown in FIG. 8) causing metatarsal implant member 810 to travel into the first metatarsal 845. It should be appreciated that metatarsal implant member 810 may be utilized in any metatarsal for restoration of the angle in the human foot. It should also be appreciated that the metatarsal implant member may be utilized for fusion of other joints in the human body.

As shown in FIG. 11, phalangeal implant member 820 is generally cylindrical in shape, has a generally solid body and extends from a spherical head portion 1105 to tapered end 1104. Spherical head portion 1105 is generally spherical in shape and has a generally hexagonal torque transmitting aperture 1108 traversing length 1110 of head portion 1105. In other non-limiting embodiments, a star-shaped aperture, a square-shaped aperture, or any other shaped aperture of any length may be utilized without departing from the scope of the invention. Torque transmitting aperture 1108 is utilized to transmit a torque from head portion 1105 to tapered end 1104 when head portion 1105 is rotated. The largest diameter 1126 of head portion 1105 is slightly larger than diameter 1032 of circular aperture 1016 on metatarsal implant member 810, which was shown previously in FIGS. 10A-B.

Head portion 1105 further has a plurality of generally flat edges 1111 (i.e., head portion 1105 has a plurality of step-like protrusions) which are provided to engage circular aperture 1016. The flat edges 1111 allow for the phalangeal implant member 820 to be coupled at a plurality of angles to metatarsal implant member 810. Each of the plurality of edges 1111 allows for a unique angle in a locked interference fit to be created by head portion 1105 within circular aperture 1016, shown in FIGS. 10A-B, as head portion 1105 of phalangeal implant member 820 is positioned within circular aperture 1016 of metatarsal implant member 810, which will be shown and described below

Further, phalangeal implant member 820 has a first smooth exterior portion 1106 extending from head portion 1105. Portion 1106 is generally cylindrical and terminates into a second generally cylindrical portion 1116, with exterior portion 1106 and cylindrical 1116 having a uniform diameter. Furthermore, cylindrical portion 1116 has a plurality of circular threads, such as threads 1118, which are circumferentially disposed on the external surface of portion 1116. Cylindrical portion 1116 may also be provided with a self-tapping leading edge 1122 to provide portion 1116 with the ability to remove bone material during insertion of the phalangeal implant member 820 into bone or other matter. It should be appreciated that the length of the phalangeal implant member 820 may be selected of varying lengths to allow a surgeon to fuse different joints (not shown). In other non-limiting embodiments, the phalangeal implant member 820 may have a continuous opening (i.e., a cannula) from torque transmitting aperture 1108 to tapered end 1104. The continuous opening or cannula may be provided to interact with a guide wire (not shown), such as a Kirschner wire, by receiving the guide wire within the continuous opening thereby positioning and locating the phalangeal implant 820.

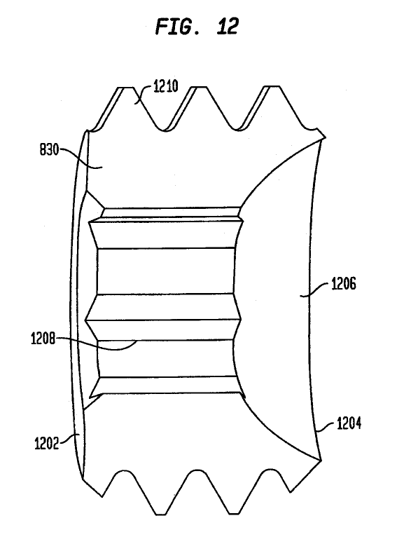

As shown in FIG. 12, intramedullary fixation assembly 800 may comprise an optional locking set screw 830 to couple the metatarsal implant member 810 to the phalangeal implant member 820. Locking set screw 830 is generally tubular in shape and extends from open first end 1202 to an open second end 1204. First end 1202 has a generally flat surface while second end 1204 has a semi-spherical groove 1206. The semi-spherical groove 1206 is provided to receive head portion 1105 of the phalangeal implant member 820 in an assembled intramedullary fixation assembly 800. Further, locking set screw 830 has a generally hexagonal torque-transmitting aperture 1208 that traverses from first end 1202 to second end 1204. In other non-limiting embodiments, a star-shaped aperture, a square-shaped aperture, or any other shaped aperture of any length may be utilized without departing from the scope of the invention.

Torque transmitting aperture 1208 is utilized to receive a torque shaped tool in order to rotate and couple locking set screw 830 to first metatarsal implant member 810 (not shown). Locking set screw 830 is also provided with a plurality of substantially similar circumferential threads 1210 in order to engage the plurality of threads 1028 on the interior surface 1026 of the metatarsal implant member 810 (shown in FIGS. 10A and 10B) and threadably couple (i.e., mechanically couple) the locking set screw 830 to the metatarsal implant member 810, thereby preventing the phalangeal implant member 820 from backing out of circular aperture 1016 on metatarsal implant member 810 and losing compression.

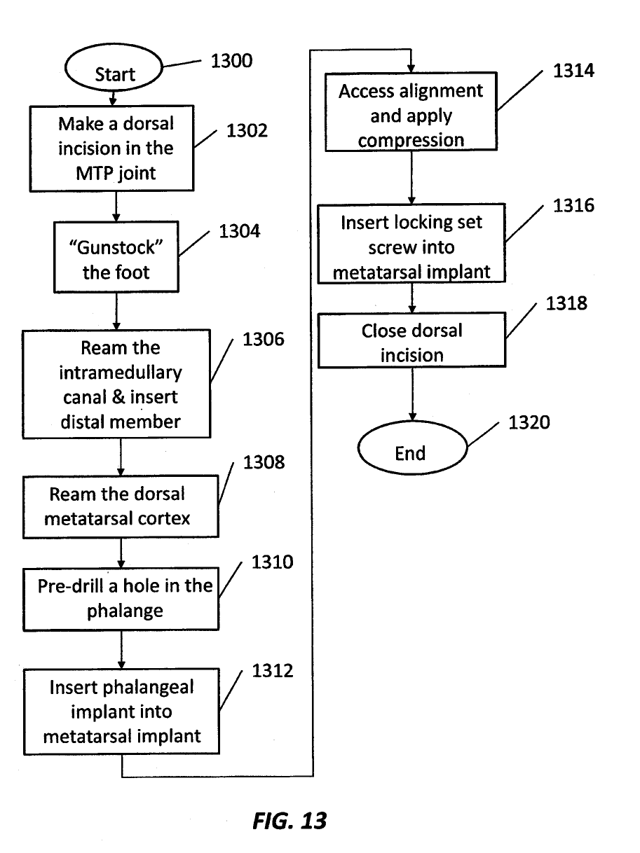

In operation, and as shown in FIGS. 8 and 13, the intramedullary fixation assembly 800 may be utilized to reconstruct an arch and/or angle in a metatarsal phalangeal joint of a human foot 840. As shown, the method starts in step 1300 and proceeds to step 1302, whereby a dorsal incision is made in the metatarsal phalangeal (MTP) joint 855 of foot 840 in order to gain access to the MTP joint 855. In step 1304, the joint capsule is separated by “Gunstocking” foot 840 (i.e., the foot 840 is bent at MTP joint 855) to expose the articular surfaces of the metatarsal 845 and first proximal phalange 850. The articulating cartilage is removed by denuding the cartilage in the MTP joint 855. Next, in step 1306, the intramedullary canal of the metatarsal 845 is reamed by drilling the metatarsal intramedullary canal and the metatarsal implant member 810 is inserted. The cylindrical portion 1018 of the metatarsal implant member 810 is inserted first into the intramedullary canal (of the metatarsal 845 to a predetermined depth until end 1002 is oriented at the opening of the MTP joint 855. In other non-limiting embodiments, the metatarsal implant member 810 may be inserted by impaction, by press fit, by reaming a hole in the intramedullary canal (not shown) or any other similar strategy or technique.

Next, in step 1308, the dorsal metatarsal cortex (not shown) is drilled and reamed to allow access to metatarsal implant member 810 from an anterior grade access. In step 1310, a cannulated drill or guide wire is used to pre-drill a pilot hole through the articular surface of the phalange 850. In step 1312, the phalangeal implant member 820 is inserted into the metatarsal implant member 810 and into the pre-drilled pilot hole by inserting the tapered end 1104 (shown in FIG. 11) into the circular aperture 1016 (shown in FIG. 10A-B) at surface 1022 and until the tapered end 1104 emanates from circular aperture 1016. Further, the phalangeal implant member 820 is inserted into the phalange 850 (shown in FIG. 8). Next in step 1314, the phalangeal implant member 820 is aligned and angle 845 (shown in FIG. 9) is formed and compression is applied to the intramedullary fixation assembly 800 by rotating the phalangeal implant member 820. The phalangeal implant member 820 is fixed at angle 845 (shown in FIG. 9). In optional step 1316, the locking set screw 830 is inserted into metatarsal implant member 810 to lock angle 845 (shown in FIG. 9) in place. Next, in step 1318, the dorsal incision is closed. The method ends in step 1320.

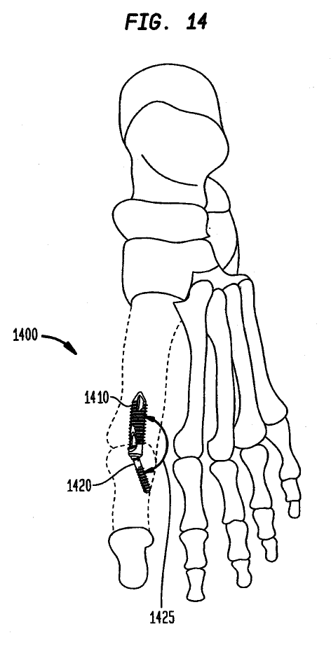

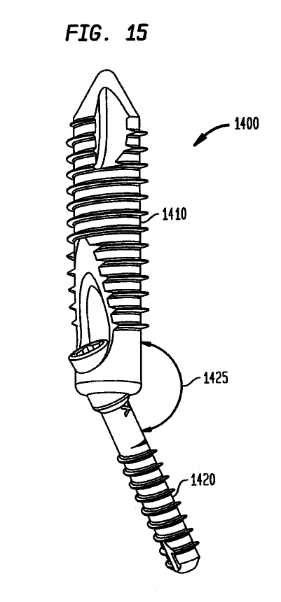

In an alternate embodiment, as shown in FIGS. 14 and 15, intramedullary fixation assembly 1400 is provided so that metatarsal implant member 1410 resides at a constant and fixed angle 1425 with phalangeal implant member 1420. The fixed angle 1425 may vary between 15 and 30 degrees and may be selected by, in one example, a surgeon to provide for the internal fixation of the bones in the human foot.

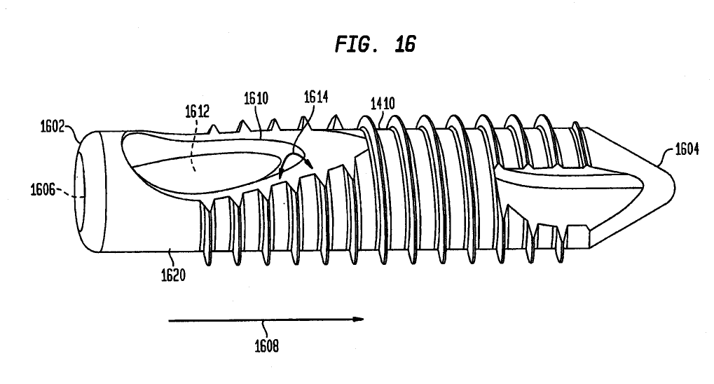

The metatarsal implant member 1410, shown in FIG. 16, is substantially similar to the distal member 140 shown and described in a previous embodiment in FIGS. 3A and 3B, and is generally tubular in shape and tapers from a first end 1602 to a second end 1604 (i.e. end 1602 has a diameter that is slightly larger than diameter of end 1604). However, in another non-limiting embodiment, metatarsal implant member 1410 has a constant width from first end 1602 to second end 1604. Further, first end 1602 has an internal circular aperture 1606 partially traversing metatarsal implant member 1410 along direction 301. Additionally, metatarsal implant member 1410 has a longitudinal aperture 1612 emanating from surface 1610, such that aperture 1612 forms a slope 1614 from in portion 1620. Slope 1614 determines the angle for restoration of the bones in a foot when phalangeal implant member 1420 (not shown) is coupled to metatarsal implant member 1410 and locks the phalangeal implant member at the fixed angle.

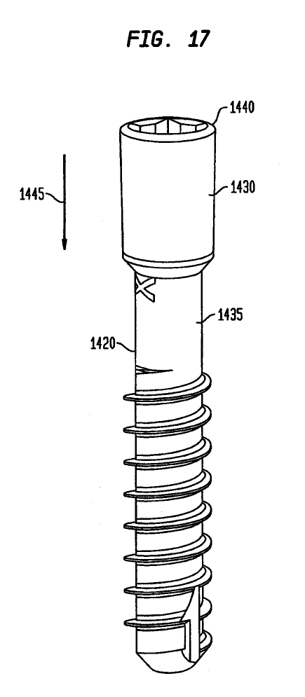

Also, the phalangeal implant member 1420, shown in FIG. 17, is substantially similar to the proximal screw member 130 that was shown in a previous embodiment, however, phalangeal implant member 1420 includes bulbous portion 1430 having a constant diameter. In other non-limiting embodiments, bulbous portion 1430 may include a morse taper (i.e., the diameter decreases from end 1440 in direction 1445) The bulbous portion 1430 is provided to be received inside aperture 1606 so that phalangeal implant member 1420 resides at a fixed angle with respect to metatarsal implant member 1410.

It should be appreciated that a plurality of intramedullary fixation assemblies, such as intramedullary fixation assembly 800, may be inserted into any of the metatarsal and phalangeal bones of a foot 840 in order to restore the natural anatomical shape of the foot 840. It should also be appreciated that the intramedullary fixation assembly 800 is delivered through a dorsal incision, thereby reducing the disruption to the plantar tissues and/or the metatarsal heads while at the same time minimizing the tension on the skin. This allows for improved wound closure, reduced operating room time, reduction in the number of incisions required and reduction in the total length of incisions. It should also be appreciated that the intramedullary fixation assembly 800 may also be utilized to restore any of the other bones in the human body. It should also be appreciated that in other non-limiting embodiments, the intramedullary assembly 800 may be utilized with graft material (i.e., autograft, allograft or other biologic agent).

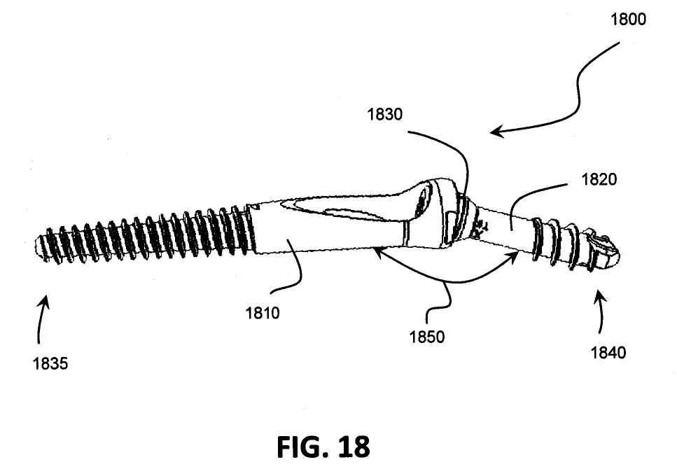

In an alternate embodiment, as shown in FIG. 18, an intramedullary fixation assembly 1800 may comprise three interconnected members for the internal fixation of, in one example, the first metatarsal bone (not shown) to the first proximal phalange bone (not shown) in the human foot. In other non-limiting embodiments, the intramedullary fixation device may be utilized for the internal fixation of the mid-foot joint (i.e., tarsometarsal joint), including fixation of the medullary canals of the metatarsal bone, medial cuneiform bone, navicular bone and talus bone. Referring back to FIG. 18, the interconnected members of the intramedullary fixation assembly 1800 may be inserted into, in one non-limiting example, the medullary canals of the first metatarsal (not shown) and the first proximal phalange (not shown) in order to create a locking torque to fix the position of the intramedullary fixation assembly 1800 within these metatarsal and proximal phalange bones. Particularly, the intramedullary fixation assembly 1800 may comprise a metatarsal implant member 1810, a lag screw member 1820 and a spherical ring 1830 for locking the metatarsal implant member 1810 to the lag screw member 1820. The metatarsal implant member 1810 is provided at the proximal end 1835, the lag screw member 1820 is provided on the distal end 1840 of the intramedullary fixation assembly 1800 and the spherical ring 1830 is provided for connecting the metatarsal implant member 1810 to the lag screw member 1820. The spherical ring 1830 increases friction between metatarsal implant member 1810 and lag screw member 1820 and allows for selectively determining the angle 1850 for positioning the metatarsal implant member 1810 with respect to the lag screw member 1820, thereby locking the metatarsal implant member 1810 to the lag screw member 1820 at the predetermined angular position as required by a surgeon as will be shown and described below. It should be appreciated that the angle 1850 initially determines the angle for restoration and may be any angle greater than 100 degrees and less than 260 degrees. It should also be appreciated that intramedullary fixation assembly 1800 may be made from a Titanium material, although, in other non-limiting embodiments, intramedullary fixation assembly 1800 may be made from SST, PEEK, NiTi, Cobalt chrome or other similar types of materials.

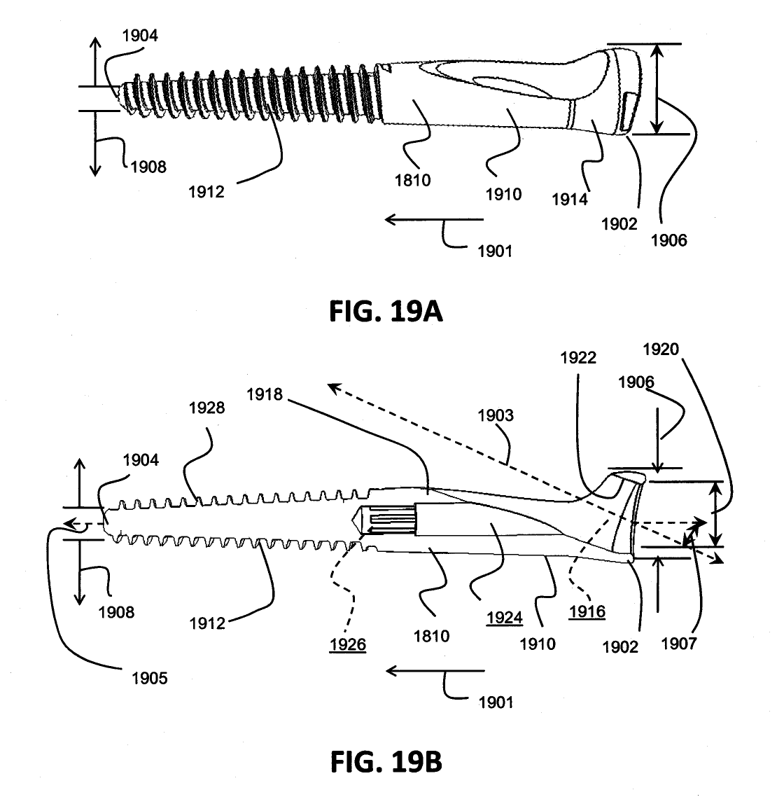

As shown in FIGS. 19A-19B, metatarsal implant member 1810 is generally tubular in shape and tapers from a first end 1902 to a second end 1904 (i.e. metatarsal implant member 1810 is generally cone-shaped with end 1902 having a diameter 1906 that is generally slightly larger than diameter 1908 at end 1904). However, in another non-limiting embodiment, metatarsal implant member 1810 has a constant width from first end 1902 to second end 1904. Metatarsal implant member 1810 comprises a generally smooth tubular portion 1910 emanating from end 1902 and terminating into a solid threaded portion 1912. Portion 1910 has a generally broader conical section 1914 at end 1902 and gets narrower along direction 1901.

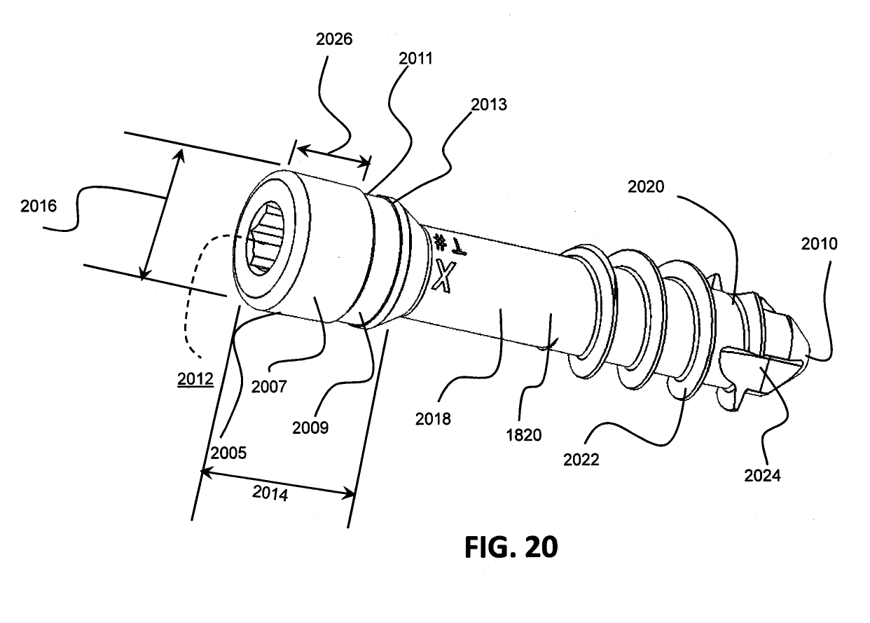

Referring now to FIG. 19B, tubular portion 1910 has an internal aperture 1916 emanating from first end 1902 and traversing tubular portion 1910 along axis 1903, which is offset from horizontal axis 1905 at an angle 1907. Circular aperture 1916 being aligned along axis 1903 causes aperture 1916 to be generally tapered and emanate from surface 1918 of tubular portion 1910, thereby causing interior surface 1922 to be generally aligned along axis 1903. Further, circular aperture 1916 has an internal diameter 1920 at end 1902, which is slightly smaller than external diameter of spherical ring 1830 (FIG. 18). The internal diameter 1920 is provided to receive spherical ring 1830 (FIG. 18) and cylindrical head portion 2005 of lag screw member 1820 (FIG. 20) in order to create a tapered frictional fit with spherical ring 1830, which will be shown and described below. Tubular portion 1910 also includes a second internal aperture 1924 emanating from first end 1902 and traversing tubular portion 1910 along direction 1901 and terminating into a hexagonal shaped aperture 1926, which is partially disposed in tubular portion 1910. The hexagonal shaped aperture 1926 is provided to receive a complementary instrument (not shown) to transmit torque from the instrument to the metatarsal implant member 1810. In other non-limiting embodiments, a star-shaped aperture, a square-shaped aperture, or any other shaped aperture may be utilized.

Further, and as shown in FIG. 19B, aperture 1926 terminates into solid threaded portion 1912. Also, threaded portion 1912 has a plurality of substantially similar circumferential threads, such as threads 1928, which are circumferentially disposed on the external surface of portion 1912. It should be appreciated that plurality of circumferential threads, such as threads 1928, are provided so that rotating metatarsal implant member 1810 causes the plurality of circumferential threads 1928 to grip or catch the medullary canal of first metatarsal bone (not shown) causing metatarsal implant member 1810 to travel into the first metatarsal bone. It should be appreciated that metatarsal implant member 1810 may be utilized in any of the other rays in the foot for applying compression and for restoration of the angle in the human foot. It should also be appreciated that the metatarsal implant member 1810 may be utilized for fusion of other joints in the human body. It should be appreciated that the length of the metatarsal implant member 1810 may be selected of varying lengths to allow a surgeon to utilize intramedullary fixation assembly 1800 to fuse different joints.

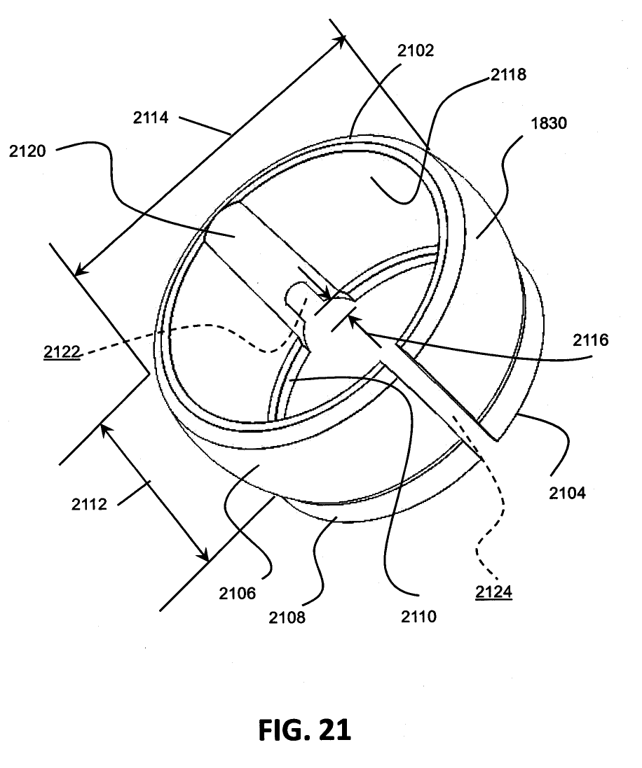

As shown in FIG. 20, lag screw member 1820 is generally cylindrical in shape, has a generally tubular body and extends from a cylindrical head portion 2005 to tapered end 2010. Cylindrical head portion 2005 is generally cylindrical in shape and includes a circumferentially ridged section 2007 terminating into a circumferentially grooved section 2009. Circumferentially grooved section is recessed in head portion 2005 and is bounded by edges 2011 and 2013. The ridged section 2007 has a width 2026 and cooperates with grooved section 2009 to receive spherical ring 1830 (FIGS. 18, 21) in order to contain lag screw member 1820 in metatarsal implant member 1810 (FIGS. 19A-B) by increasing friction between the spherical ring 1830 (FIGS. 18, 21), lag screw member 1820 and metatarsal implant member 1810 (FIG. 18). Further, lag screw member 1820 has a generally hexagonal torque transmitting aperture 2012 traversing length 2014 of head portion 2005. In other non-limiting embodiments, a star-shaped aperture, a square-shaped aperture, or any other shaped aperture of any length may be utilized without departing from the scope of the invention. Torque transmitting aperture 2012 is utilized to transmit a torque from head portion 2005 to tapered end 2010 when head portion 2005 is rotated. Diameter 2016 of head portion 2005 is slightly smaller than internal diameter 1920 of circular aperture 1916 on metatarsal implant member 1810, which was shown previously in FIGS. 19A-B.

Further, head portion 2005 terminates into a first smooth tubular portion 2018. Portion 2018 is generally cylindrical, is cannulated and terminates into a second generally cylindrical portion 2020. Portion 2020 is also cannulated, with tubular portion 2018 and portion 2020 having a uniform diameter. The lag screw member 1820 may have a continuous opening (i.e., a cannula) from torque transmitting aperture 2012 to tapered end 2010. The continuous opening or cannula may be provided to interact with a guide wire (not shown), such as a Kirschner wire, by receiving the guide wire within the continuous opening thereby positioning and locating the lag screw member 1820. In other non-limiting embodiments, the lag screw member 1820 is solid. Furthermore, cylindrical portion 2020 has a plurality of circular threads, such as threads 2022, which are circumferentially disposed on the external surface of portion 2020. Cylindrical portion 2020 may also be provided with a self-tapping leading edge 2024 to provide portion 2020 with the ability to remove bone material during insertion of the lag screw member 1820 into bone or other matter. It should be appreciated that the length of the lag screw member 1820 may be selected of varying lengths to allow a surgeon to utilize intramedullary fixation assembly 1800 to fuse different joints.

Referring now to FIG. 21, intramedullary fixation assembly 1800 may comprise spherical ring 1830 to couple the metatarsal implant member 1810 (FIG. 18) to the lag screw member 1820 (FIG. 18) and provide for creating a locking torque as the lag screw member 1820 is pulled deeper into the spherical ring 1830. The initial position of the lag screw member 1820 may be selectively adjusted by rotating the lag screw member 1820, which is coupled to the spherical ring 1830, within the metatarsal implant member 1810. As shown, spherical ring 1830 is generally circular in shape and extends from open first end 2102 to an open second end 2104.

Spherical ring 1830 has a first portion 2106 having a convex cross-section and terminates into a second portion 2108 having an “L-shaped” cross-section (i.e., second portion 2108 has a raised edge 2110 on its interior). Second portion 2108 is provided to reside within tapered aperture 1916 (FIG. 19B) and forms a tapered fit within the aperture 1916. First portion 2106 has height 2112. Further spherical ring 1830 has an internal diameter 2114, which is substantially the same as the internal diameter 1920 of lag screw member 1830 (FIG. 19B). Also, second portion 2108 is generally circular in shape and has height 2116, which is generally smaller than width of grooved section 2009 (FIG. 20). The raised edge 2110 is provided to be received in grooved section 2009, with edge 2110 being restrained by edges 2011 and 2013 (FIG. 20) causing spherical ring 1830 to expand outward and increase the contact pressure between the spherical ring 1830 and the metatarsal implant member 1810 as the lag screw member 1830 is driven further into the underlying bone.

Also as shown in FIG. 21, spherical ring 1830 includes a vertical groove 2120 traversing its interior 2118 and which terminates into a “Key-hole” shaped slot 2122 partially disposed in the spherical ring 1830. Spherical ring 1830 also comprises a channel 2124 formed in portions 2106 and 2108 and located diametrically opposite the vertical groove 2120. The vertical groove 2120 cooperates with channel 2124 and “key-hole” shaped slot 2122 to allow the spherical ring 1830 to expand outward inside the tapered aperture 1916 (FIG. 19B) as the lag screw member 1820 is placed in tension with the metatarsal implant member 1810 by pulling the lag screw member 1820 deeper into the spherical ring 1830.

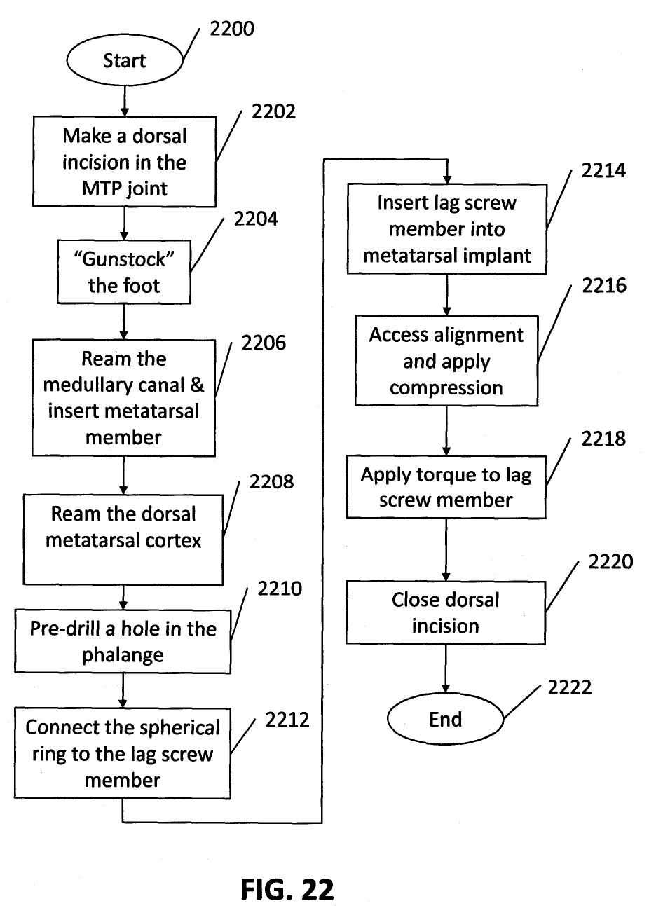

In operation, and as shown in FIGS. 18-22, the intramedullary fixation assembly 1800 may be utilized to compress, in one example, the metatarsal phalangeal joint (not shown). As shown, the method starts in step 2200 and proceeds to step 2202, whereby a dorsal incision is made above the metatarsal phalangeal (MTP) joint of the foot in order to gain access to the MTP joint. In step 2204, the joint capsule is separated by “Gunstocking” the foot (i.e., the foot is bent at MTP joint) to expose the articular surfaces of the first metatarsal bone and first proximal phalange bone. The articulating cartilage is removed by denuding the cartilage in the MTP joint. Next, in step 2206, the intramedullary canal of the metatarsal bone is reamed by drilling the metatarsal intramedullary canal and the metatarsal implant member 1810 is inserted. The threaded portion 1912 (FIG. 19A) of the metatarsal implant member 1810 is inserted first into the intramedullary canal of the first metatarsal to a predetermined depth until end 1902 is oriented at the opening of the MTP joint. In other non-limiting embodiments, the metatarsal implant member 1810 may be inserted by impaction, by press fit, by reaming a hole in the intramedullary canal (not shown) or any other similar strategy or technique.

Next, in step 2208, the dorsal metatarsal cortex (not shown) is drilled and reamed to allow access to metatarsal implant member 1810 from an anterior grade access. In step 2210, a cannulated drill or guide wire is used to pre-drill a pilot hole through the articular surface of the phalange. In step 2212, the spherical ring 1830 is coupled to the lag screw member 1820 and, in step 2214, the lag screw member 1820 is inserted into the metatarsal implant member 1810 and into the pre-drilled pilot hole by inserting the cylindrical portion 2020 (shown in FIG. 20) into the internal aperture 1916 at surface 1918 (shown in FIG. 19B) until the cylindrical portion 2020 emanates from internal aperture 1916. Further, the lag screw member 1820 is inserted into the first proximal phalange. Next in step 2216, the lag screw member 1820 is aligned at angle 1850 (FIG. 18) and the lag screw member 1820 is driven into the first proximal phalange. The tension between the first metatarsal and the first proximal phalange bone pulls the bones together. This compresses the MTP joint and creates tension within the intramedullary fixation assembly 1800. Next, in step 2218, torque is applied to the lag screw member 1820 to create additional tension and pull the lag screw member 1820 deeper into the spherical ring 1830. The tapered fit between the spherical ring 1830 and portion 2005 (FIG. 20) causes the spherical ring 1830 to expand outward as the lag screw member 1820 is pulled deeper into internal aperture 1916 (FIG. 19). This increases contact pressure and therefore, friction, between the first portion 2106 (FIG. 21) and interior surface 1922 (FIG. 19B), creating a locking torque that resists relative motion between the spherical ring 1830 and metatarsal implant member 1810 and fixing the position of the lag screw member 1820 with the metatarsal implant member 1810. Next, in step 2220, the dorsal incision is closed. The method ends in step 2222.

It should also be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. An intramedullary fixation assembly for bone fixation, comprising:
an implant member positioned at a proximal end of the intramedullary fixation assembly;
a lag screw member positioned at a distal end of the intramedullary fixation assembly, and
a locking member coupled to the lag screw member;
wherein the lag screw member is configured for coupling with the implant member at an angle.

2. The intramedullary fixation assembly of claim 1, wherein the locking member locks the lag screw member at the angle.

3. The intramedullary fixation assembly of claim 2, wherein the lag screw member is locked upon compressing the lag screw member with the implant member.

4. The intramedullary fixation assembly of claim 1, wherein the implant member includes a first elongated body comprising a first threaded portion at a first end and an opening at a second end.

5. The intramedullary fixation assembly of claim 3, wherein the first threaded portion comprises a plurality of bone threads on an outer surface of the first threaded portion.

6. The intramedullary fixation assembly of claim 4, wherein the first threaded portion comprises a self-tapping edge for removal of bone material during insertion of the implant member.

7. The intramedullary fixation assembly of claim 1, wherein the implant member comprises a first internal aperture and a second internal aperture disposed in the first elongated body.

8. The intramedullary fixation assembly of claim 7, wherein the second internal aperture is partially disposed in the first elongated body along a longitudinal axis of the first elongated body.

9. The intramedullary fixation assembly of claim 8, wherein the first internal aperture is partially disposed in the first elongated body along an axis offset from the longitudinal axis.

10. The intramedullary fixation assembly of claim 7, wherein the second internal aperture comprises a hexagonally shaped opening, a star-shaped opening, or a square-shaped opening partially disposed in the first elongated body.

11. The intramedullary fixation assembly of claim 10, wherein each of the openings is provided to receive a complementary shaped member.

12. The intramedullary fixation assembly of claim 7, wherein the first internal aperture traverses the first elongated body from the second end to an exterior surface on the first elongated body.

13. The intramedullary fixation assembly of claim 7, wherein the first internal aperture is tapered.

14. The intramedullary fixation assembly of claim 7, wherein the first internal aperture forms a predetermined angle with the second internal aperture.

15. The intramedullary fixation assembly of claim 1, wherein the lag screw member comprises a second elongated body, a second threaded portion at a third end and a bulbous portion at a fourth end.

16. The intramedullary fixation assembly of claim 7, wherein the first internal aperture is provided to receive the locking member and causes an interference fit with the bulbous portion.

17. The intramedullary fixation assembly of claim 16, wherein the bulbous portion comprises a circumferential groove on an external surface of the bulbous portion.

18. The intramedullary fixation assembly of claim 16, wherein the bulbous portion includes an orifice longitudinally coextensive with a length of the bulbous portion.

19. The intramedullary fixation assembly of claim 18, wherein the orifice has a hexagonal shape, a star shape, or a square shape.

20. The intramedullary fixation assembly of claim 18, wherein the orifice is provided to receive a complementary shaped end of an instrument.

21. The intramedullary fixation assembly of claim 15, wherein the second threaded portion contains a plurality of bone threads circumferentially disposed along an outer surface of the second threaded portion.

22. The intramedullary fixation assembly of claim 15, wherein the second threaded portion includes a self-tapping edge for removing bone material during insertion of the lag screw member.

23. The intramedullary fixation assembly of claim 1, wherein the locking member is a spherical ring comprising a first circular portion having a convex cross-section coupled to a second circular portion having a uniform cross-section

24. The intramedullary fixation assembly of claim 23, wherein the second circular portion comprises a raised edge on an interior surface for causing an interference lock with the circumferential groove.

25. A method for fusing a joint, comprising:
connecting an implant member to a first bone; and
connecting a locking member to a lag screw member;
forming the medullary canals of the first bone and a second bone;
inserting the implant member into a medullary canal of the first bone;
inserting the lag screw member into the implant member;
inserting the lag screw member into a medullary canal of the second bone;
positioning the lag screw member at an angle; and
applying compression to the lag screw member causing the circular member to lock the lag screw member at the angle, thereby fusing the joint.

26. The method of claim 25, wherein the implant member includes a first elongated body comprising a first threaded portion at a first end and an opening at a second end.

27. The method of claim 26, wherein the first threaded portion comprises a plurality of bone threads on an outer surface of the first threaded portion.

28. The method of claim 26, wherein the first threaded portion comprises a self-tapping edge for removal of bone material during insertion of the implant member.

29. The method of claim 27, wherein the implant member comprises a first internal aperture and a second internal aperture disposed in the first elongated body.

30. The method of claim 29, wherein the second internal aperture is partially disposed in the first elongated body along a longitudinal axis of the first elongated body.

31. The method of claim 29, wherein the first internal aperture is partially disposed in the first elongated body along an axis offset from the longitudinal axis.

32. The method of claim 29, wherein the second internal aperture comprises a hexagonally shaped opening, a star-shaped opening, or a square-shaped opening partially disposed in the first elongated body.

33. The method of claim 32, wherein each of the openings is provided to receive a complementary shaped member.

34. The method of claim 29, wherein the first internal aperture traverses the first elongated body from the second end to an exterior surface on the first elongated body.

35. The method of claim 29, wherein the first internal aperture is tapered.

36. The method of claim 29, wherein the first internal aperture forms a fixed angle with the second internal aperture.

37. The method of claim 25, wherein the lag screw member comprises a second elongated body, a second threaded portion at a third end and a bulbous portion at a fourth end.

38. The method of claim 37, wherein the first internal aperture is provided to receive the locking member and causes an interference fit with the bulbous portion.

39. The method of claim 38, wherein the bulbous portion includes an orifice longitudinally coextensive with a length of the bulbous portion.

40. The method of claim 39, wherein the orifice has a hexagonal shape, a star shape, or a square shape.

41. The method of claim 39, wherein the orifice is provided to receive a complementary shaped end of an instrument.

42. The method of claim 37, wherein the second threaded portion contains a plurality of bone threads circumferentially disposed along an outer surface of the second threaded portion.

43. The method of claim 37, wherein the second threaded portion includes a self-tapping edge for removing bone material during insertion of the lag screw member.

44. The method of claim 25, wherein the locking member is a spherical ring comprising a first circular portion having a convex cross-section coupled to a second circular portion having a uniform cross-section.

45. The method of claim 44, wherein the second circular portion comprises a raised edge on an interior surface for causing an interference lock with the circumferential groove.

46. An intramedullary fixation system comprising:
an implant member configured for positioning at a proximal end of the intramedullary fixation assembly;
a lag screw member configured for positioning at a distal end of the intramedullary fixation assembly, and
a circular member adapted to be coupled to the lag screw member; and
an instrument adapted for forming a plurality of medullary canals, said medullary canals adapted to receive said implant member and said lag screw member;
wherein the lag screw member is configured to be coupled to the implant member at an angle; and
wherein the circular member is adapted for locking the lag screw member at the angle upon compressing the lag screw member with the implant member.

47. The intramedullary fixation assembly of claim 46, wherein the implant member includes a first elongated body comprising a first threaded portion at a first end and an opening at a second end.

48. The intramedullary fixation assembly of claim 47, wherein the first threaded portion comprises a plurality of bone threads on an outer surface of the first threaded portion.

49. The intramedullary fixation assembly of claim 48, wherein the first threaded portion comprises a self-tapping edge for removal of bone material during insertion of the implant member.

50. The intramedullary fixation assembly of claim 46, wherein the implant member comprises a first internal aperture and a second internal aperture disposed in the first elongated body.

51. The intramedullary fixation assembly of claim 50, wherein the second internal aperture is partially disposed in the first elongated body along a longitudinal axis of the first elongated body.

52. The intramedullary fixation assembly of claim 51, wherein the first internal aperture is partially disposed in the first elongated body along an axis offset from the longitudinal axis.

53. The intramedullary fixation assembly of claim 50, wherein the second internal aperture comprises a hexagonally shaped opening, a star-shaped opening, or a square-shaped opening partially disposed in the first elongated body.

54. The intramedullary fixation assembly of claim 53, wherein each of the openings is provided to receive a complementary shaped member.

55. The intramedullary fixation assembly of claim 50, wherein the first internal aperture traverses the first elongated body from the second end to an exterior surface on the first elongated body.

56. The intramedullary fixation assembly of claim 50, wherein the first internal aperture is tapered.

57. The intramedullary fixation assembly of claim 50, wherein the first internal aperture forms a predetermined angle with the second internal aperture.

58. The intramedullary fixation assembly of claim 46, wherein the lag screw member comprises a second elongated body, a second threaded portion at a third end and a bulbous portion at a fourth end.

59. The intramedullary fixation assembly of claim 50, wherein the first internal aperture is provided to receive the circular member and causes an interference fit with the bulbous portion.

60. The intramedullary fixation assembly of claim 59, wherein the bulbous portion comprises a circumferential groove on an external surface of the bulbous portion.

61. The intramedullary fixation assembly of claim 59, wherein the bulbous portion includes an orifice longitudinally coextensive with a length of the bulbous portion.

62. The intramedullary fixation assembly of claim 61, wherein the orifice has a hexagonal shape, a star shape, or a square shape.

63. The intramedullary fixation assembly of claim 61, wherein the orifice is provided to receive a complementary shaped end of an instrument.

64. The intramedullary fixation assembly of claim 58, wherein the second threaded portion contains a plurality of bone threads circumferentially disposed along an outer surface of the second threaded portion.

65. The intramedullary fixation assembly of claim 58, wherein the second threaded portion includes a self-tapping edge for removing bone material during insertion of the lag screw member.

66. The intramedullary fixation assembly of claim 46, wherein the circular locking member is a spherical ring comprising a first circular portion having a convex cross-section coupled to a second circular portion having a uniform cross-section

67. The intramedullary fixation assembly of claim 66, wherein the second circular portion comprises a raised edge on an interior surface for causing an interference lock with the circumferential groove.
